(19) United States
(12) Patent Application Publication
(10) Pub. No.: US 2012/0149895 A1
(43) Pub. Date: Jun. 14, 2012

(54) PROCESS FOR DIMETHYLATION OF ACTIVE METHYLENE GROUPS

(75) Inventors: Branko Jenko, LG (SI); Anton Copar, Ljubljana (SI)

(73) Assignee: LEK PHARMACEUTICALS D.D., Ljubjana (SI)

(21) Appl. No.: 13/262,587

(22) PCT Filed: Mar. 30, 2010
(86) PCT No.: PCT/EP10/54151

§ 371 Date: Feb. 29, 2012

(30) Foreign Application Priority Data
Apr. 1, 2009 (EP) 09157126.5

(51) Int. Cl. C07C 231/14; C07D 273/00; C07C 253/30

(52) U.S. Cl. 540/460; 558/369; 564/126

(57) ABSTRACT

The present invention discloses a process for dimethylation of active methylene groups. Specifically, the invention discloses a process for preparing 3-amino-2,2-dimethylpropanamide. Compounds produced by the present dimethylation process such as 3-amino-2,2-dimethylpropanamide can be used as intermediates in the route of synthesis of therapeutic, prophylactic or diagnostic agent, for example aliskiren or cryptophycin. Particularly, the invention relates to embodiments further extending to processes for preparing pharmaceutical dosage form comprising said therapeutic, prophylactic or diagnostic agents. More specifically, the invention relates to the use of compounds produced by the present dimethylation process for the manufacture of therapeutic, prophylactic or diagnostic agents or for the manufacture of pharmaceutical dosage forms comprising said therapeutic, prophylactic or diagnostic agents. The processes according to the present invention can be beneficially applied for the synthesis of various active pharmaceutical ingredients, such as aliskiren or crypthophycin.

### FIELD OF THE INVENTION

The present invention relates to a process for dimethylation of active methylene groups. The invention further relates to a process for preparing 3-amino-2,2-dimethylpropanamide. Compounds produced by the present dimethylation process can be used as intermediates in the route of synthesis of therapeutic, prophylactic or diagnostic agent, for example aliskiren or cryptophycins. Particularly, the present invention relates to embodiments further extending to processes for preparing the pharmaceutical dosage form comprising said therapeutic, prophylactic or diagnostic agents. The invention also relates to the use of compounds produced by the present dimethylation process for the manufacture of therapeutic, prophylactic or diagnostic agents or for the manufacture of pharmaceutical dosage forms comprising said therapeutic, prophylactic or diagnostic agents. The processes according to the present invention can be beneficially applied for the synthesis of various active pharmaceutical ingredients, such as aliskiren, crypthophycin and other compounds alike.

### BACKGROUND OF THE INVENTION

Compounds comprising dimethylated methylene groups are important intermediates for active pharmaceutical ingredients. There is a great interest in obtaining a process for dimethylation of active methylene groups that provides a product in high yield with little or no monomethyl and desmethyl impurities, being rapid, simple, robust, relatively nonhazardous, and suitable for industrial scale. The absence of monomethyl and desmethyl impurities is of immense importance, since their removal from the product is very burdensome and causes many methods to have consequently unsatisfied yields.

The process for preparing compounds like α,α-dimethyl substituted carboxylic derivatives was disclosed in Chem. Pharm. Bull. 33, 3046 (1985), where ethyl cyanoacetate was methylated by using methyl iodide in the presence of potassium hydroxide in ethanol. Similarly Tetrahedron 44, 1107 (1988) discloses dimethylation of alkyl cyanoacetate by using methyl iodide in the presence of sodium hydride in tetrahydrofuran. Tetrahedron Lett. 46, 6337 (2005) further elucidates the use of methyl iodide with sodium ethoxide in ethanol. All three routes of synthesis suffer from substantial presence of monomethyl and desmethyl impurities in the product. The 2005 publication suggests solving said drawback by implementing N-CBz protection with further purification, but the derivatisation lowers the total yield.

Further dimethylation processes for CH-acidic compounds employing the conventional methylation agents methyliodide or -bromide, wherein potassium carbonate is used as the base and dimethyl sulfoxide, dimethyl formamide or a mixture of dimethyl sulfoxide and tetrahydrofuran is used as the solvent, are disclosed e.g. in: EP 1 717 238 A1; K. Beck et. al., Chemische Berichte, Vol. 120, 1987, pages 477 to 483; W. Adam, Synthesis, 1995, pages 1163 to 1170; WO 2008/147697 and DE 103 57 978 A1.

An attempt to use much cheaper dimethyl sulphate in dimethylation process of 2-cyanoacetamide was disclosed in CN 1990461, but the procedure is low reproducible as a considerable amount of monomethyl residue has been detected.

Dimethylation of active methylene groups has been further disclosed in WO 00/023429, where dimethylation of ethyl 2-cyanoacetate was achieved by using methyl iodide and caesium carbonate in dimethylformamide.

EP 0 924 196 A1 describes a process for alkylation of alkyl- or benzylcyano derivatives in the presence of trialkylamines or -phosphines. Among others, this document discloses the dimethylation of benzyl cyanide in aqueous sodium hydroxide in the presence of trioctylamine, wherein methyl chloride is used as the methylation agent at elevated pressure. However, since this method uses extremely caustic conditions, it is not applicable to hydrolysable starting compounds.

Therefore, the object of the present invention is to provide an improved process for dimethylation of active methylene groups.

### SUMMARY OF THE INVENTION

Various aspects, advantageous features and preferred embodiments of the present invention as summarized in the following items, respectively alone or in combination, contribute to solving the object of the invention:

(1) A process for preparing a compound of the formula (I):

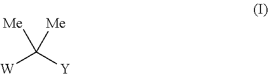

in which W denotes an electron withdrawing group having −M-effect,
Y is the same or different electron withdrawing group as W, or Y is selected from groups having +M-effect or no M-effect, except H,
wherein a compound of formula (II):

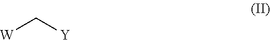

in which W and Y are defined as above, is reacted with methyl chloride in the presence of a proton acceptor in a solvent essentially consisting of a polar aprotic solvent or a mixture of a polar aprotic solvent and non-polar aprotic solvent.

The term “electron withdrawing group” as used herein means moieties having a polar electronic effect defined by a negative mesomeric effect (so called −M-effect). Preferably, W additionally has a negative inductive effect (so called −I-effect), i.e. preferably both −I-effect and −M-effect. Thereby, electrostatic forces are modified in the methylene group located between the two W groups of a compound of formula (II), namely the electrons are drawn away from the methylene group. This in turn promotes an abstraction of the H-atoms of the methylene group in form of protons, i.e. there is a kind of “C—H acidity”. Therefore, this kind of methylene groups may be referred to as “active methylene group”.

When the group Y is not an electron withdrawing group as defined above, it can be selected in view of the other group W of formula (II) with the proviso that the acidity of the protons of the linking methylene group between W and Y is set such that its methylene protons acidity is sufficient to enable substantial dimethylation, that is dimethylation reaction affording conversion of compound of formula (II) to compound of formula (I) of at least 50%, preferably at least 80%, more preferably at least 90% and in particular at least 99%. Examples for “Y groups having +M-effect or no M-effect except H (hydrogen)” are groups having −I-effect and +M-effect, +I-effect and +M-effect, −I-effect only or +I effect only. Azido, NHCOOR, SOR′, OR′ and SR′, wherein R and R′ are defined below, represent examples for groups having −I-effect and +M-effect. Aryl groups selected from a single six-membered ring or condensed six-membered rings, such as phenyl or naphtyl, are examples for groups having −I-effect and +M-effect. Unsubstituted linear or branched alkyl groups e.g. represent groups having +I-effect only. −NR₃⁺ wherein R is defined as above and −NH₃⁺ e.g. represent groups having −I-effect only. If Y is selected from the groups having +M-effect the acidity is sufficient to enable substantial dimethylation, that is dimethylation reaction affording conversion of compound of formula (II) to compound of formula (I) of at least 50%, preferably at least 80%, more preferably at least 90% and in particular at least 99% only if +M-effect is annulled by −I-effect and/or by strong electron withdrawing properties of group W.

According to this beneficial aspect of the invention, advantageous reaction conditions are provided which enable a better reactivity of MeCl over MeI, since the solvent does essentially contain no water or other polar solvents. However, it is known that organic solvents may contain minute or still small amounts of water under normal handling conditions. In order to provide an efficient process, the amount of water in said solvents should be kept below 5 percent by weight based on the mass of the solvent.

(2) A process for preparing a compound of the formula (I):

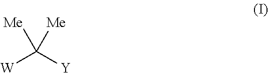

in which W denotes an electron withdrawing group having −M-effect,
Y is the same or different electron withdrawing group as W, or Y is selected from groups having +M-effect or no M-effect, except H,
wherein a compound of formula (II):

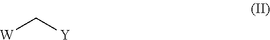

in which W and Y are defined as above, is reacted with methyl chloride in the presence of a proton acceptor in the absence of a solvent.

According to this alternative aspect of the invention, a dimethylation process is provided wherein no solvent is needed. Thus, the process is especially advantageous in view of environmental friendliness, working conditions and possibly economy.

As to the meanings of “electron withdrawing group” and “groups having +M-effect or no M-effect, except H”, reference is made to the explanations under item (1) above.

(3) The process according to item (1) or (2), wherein W is selected from the group consisting of:

### CN, CHO and NO₂;

COOR, CONH₂, CONHR, CONR₂, COSR, CSOR, CSNH₂, CSNHR and CSNH₂, wherein R is substituted or unsubstituted alkyl, substituted or unsubstituted aryl, substituted or unsubstituted arylalkyl, substituted or unsubstituted heteroaryl or substituted or unsubstituted heteroarylalkyl; and
COR′, SO₂R′, CR′═NR″, wherein R′ and R″ are independently selected from the group consisting of substituted or unsubstituted alkyl, substituted or unsubstituted aryl, substituted or unsubstituted arylalkyl, substituted or unsubstituted heteroaryl or substituted or unsubstituted heteroarylalkyl;
or W and Y cooperatively represent a group of the formula Z′(CH₂)ₚZ″, wherein Z′ and Z″ are the same or different and are either CO, CO—O—, CO—NR*-, CO—S—, and SO₂ group, wherein R* is H, substituted or unsubstituted alkyl, substituted or unsubstituted aryl, substituted or unsubstituted arylalkyl, substituted or unsubstituted heteroaryl or substituted or unsubstituted heteroarylalkyl, and p is an integer between 1 and 4; and
Y is the same or different electron withdrawing group selected from W defined above, or Y is selected from the group consisting of azido, substituted or unsubstituted aryl, substituted or unsubstituted alkyl, NHCOOR, SOR′, OR′ and SR′, preferably azido, substituted or unsubstituted aryl and substituted or unsubstituted alkyl, wherein R and R′ are selected from the group consisting of substituted or unsubstituted alkyl, substituted or unsubstituted aryl, substituted or unsubstituted arylalkyl, substituted or unsubstituted heteroaryl or substituted or unsubstituted heteroarylalkyl.

As used herein, “alkyl” means straight or branched alkyl of 1 to 10 carbon atoms, preferably 1 to 8 carbon atoms and more preferably 1 to 6 carbon atoms, “cycloalkyl” means cycloalkyls of 3 to 8 carbon atoms, “aryl” means substituted or unsubstituted aryls selected from a single six-membered ring or condensed six-membered rings, preferably phenyl or naphtyl, more preferably phenyl, “arylalkyl” means substituted or unsubstituted phenylalkyl, where alkyl is 1 to 6 carbon atoms, “heteroaryl” means aromatic rings of 5 to 7 carbon atoms where 1, 2 or 3 carbon atoms are exchanged by oxygen, nitrogen or sulphur, and “heteroarylalkyl” means the aforementioned heteroaryls comprising alkyl of 1 to 6 carbon atoms. Any aforementioned alkyl, aryl, arylalkyl or heteroarylalkyl can be optionally unsaturated in its alkyl moiety, or substituted in its aromatic and/or alkyl moiety with one or more substituents selected from alkyl of 1 to 4 carbon atoms, F, Cl, Br, OH, OCH₃, CF₃, and COOR¹, where R¹ is H, alkyl of 1 to 4 carbon atoms, phenyl, alkenyl or alkynyl of 2 to 10 carbon atoms.

(4) The process according to any one of the preceding items, wherein W is selected from the group consisting of:

### CN and NO₂;

COOR, CONH₂, CONHR, CONR₂, COSR, CSOR, CSNH₂, CSNHR, CSNR₂ and COR, wherein R is substituted or unsubstituted alkyl, substituted or unsubstituted aryl, substituted or unsubstituted arylalkyl, substituted or unsubstituted heteroaryl or substituted or unsubstituted heteroarylalkyl; and
Y is same or different electron withdrawing group selected from W defined above.

(5) The process according to any one of the preceding items, wherein W is selected from a group consisting of:

### COOR, CONH₂, CONHR, CONR₂, COSR, CSOR, CSNH₂, CSNHR, CSNR₂, and COR,

wherein R is substituted or unsubstituted alkyl, substituted or unsubstituted aryl, substituted or unsubstituted arylalkyl, substituted or unsubstituted heteroaryl or substituted or unsubstituted heteroarylalkyl.

(6) The process according to any one of the preceding items, wherein Y is the same electron withdrawing group as W.

(7) The process according to any one of the preceding items, wherein W is selected from a group consisting of: COOR, CONH₂, CONHR, CONR₂, COSR, CSOR, CSNH₂, CSNHR and CSNR₂ wherein R is substituted or unsubstituted alkyl, substituted or unsubstituted aryl, substituted or unsubstituted arylalkyl, substituted or unsubstituted heteroaryl or substituted or unsubstituted heteroarylalkyl, and Y is CN.

(8) The process according to any one of the preceding items, wherein W is CN and Y is COOR, CONH₂, CONHR or CONR₂, wherein R is substituted or unsubstituted alkyl, preferably methyl or ethyl.

(9) The process according to item (8), wherein Y is preferably COOR.

(10) The process according to any one of the preceding items, wherein W is CN and Y is selected from the group consisting of COOR, CONH₂, CONHR, CONR₂, COSR, CSOR, CSNH₂, CSNHR, CSNR₂ and COR, wherein R is substituted or unsubstituted alkyl, substituted or unsubstituted aryl, substituted or unsubstituted arylalkyl, substituted or unsubstituted heteroaryl or substituted or unsubstituted heteroarylalkyl.

(11) The process according to any one of the preceding items, wherein W is CN and Y is COOR wherein R is substituted or unsubstituted alkyl or benzyl, preferably methyl, ethyl or benzyl, more preferably methyl or ethyl.

(12) The process according to any one of the preceding items, wherein the proton acceptor is selected from the group consisting of alkali metal carbonate, preferably lithium, sodium, cesium or potassium carbonate, more preferably cesium carbonate or potassium carbonate, and in particular potassium carbonate.

(13) The process according to any one of items (1) and (3) to (12), wherein the polar aprotic solvent is selected from the group consisting of sulfoxides, sulphones and amides, preferably from DMSO and DMF, more preferably DMF.

(14) The process according to any one of items (1) and (3) to (13), wherein the non-polar aprotic solvent comprised in the mixture of a polar aprotic solvent and non-polar aprotic solvent is selected from the group consisting of acetonitrile, ethers and C₅-C₂₀ hydrocarbons, preferably acetonitrile, diethylether, THF, pentane and hexane.

(15) The process according to any one of items (1) and (3) to (14), wherein the solvent essentially consisting of the mixture of a polar aprotic solvent and non-polar aprotic solvent has a volume ratio of polar aprotic solvent to aprotic solvent of 1:0 to 1:2, preferably the ratio is be selected with the proviso that sufficient solubility of a proton acceptor is provided.

(16) The processes according to any one of items (1) and (3) to (15), wherein the solvent essentially consisting of the polar aprotic solvent or the mixture of the polar aprotic solvent and non-polar aprotic solvent is used in a mass ratio of solvent to compound of formula (II) of about 1 to 20, preferably about 1 to 5, and more preferably about 2 to 3.

The word “about” used herein means that the value it precedes can vary for 20% of the value, preferably 10% of the value, more preferably it means together with the value it precedes exactly that value.

(17) The process according to any one of items (1) and (3) to (15), wherein said solvent is used in a mass ratio of solvent to compound of formula (II) of 1 or less, preferably 0.3 or less, more preferably 0.1 or less.

(18) The process according to any one of the preceding items, wherein the compound of formula (II) is of liquid or fluid nature, preferably of liquid nature in case the process is carried out in the absence of solvent.

(19) The process according to any one of items (1) to (19), wherein methyl chloride is provided in gaseous or fluid form, preferably in gaseous form in case the process is carried out in the presence a solvent or in fluid form in case the process is carried out in the absence of solvent.

(20) The process according to any one of the preceding items, wherein the reaction is carried out at atmospheric pressure or elevated pressure, preferably at pressures from about 1 to about 3 bars, more preferably at atmospheric pressure.

(21) The process according to any one of the preceding items, wherein the reaction is carried out at atmospheric pressure at a temperature from about −10° C. to about 100° C., preferably from about 15 to about 35° C. at atmospheric pressure, or wherein the reaction is carried out at elevated pressures at a temperature below about 10° C., preferably below about 5° C., more preferably below about 0° C.

(22) The process according to any one of the preceding items, wherein the reaction is stopped when the concentration of monomethylated intermediate compound is below 1 area-% compared to compound of formula (II), preferably below 0.1 area-%, and more preferably below the limit of detection in gas chromatogram.

(23) The processes according to item (22), wherein remaining methyl chloride is removed after reaction by heating the reaction mixture or bubbling with inert gas, preferably by bubbling with inert gas.

(24) The process according to any one of the preceding items, further comprising a subsequent step of converting W and/or Y being an ester group to amide group.

(25) The process according to any one of the preceding items, further comprising a subsequent step of converting W and/or Y being COOR, CONH₂, CONHR, CONR₂, COSR, CSOR, CSNH₂, CSNHR or CSNR₂ to COOH, CONH₂, COSH, CSOH or CSNH₂, respectively.

(26) The process according to any one of the preceding items, further comprising the subsequent step of converting W and/or Y being a cyano group to aminomethyl group (—CH₂—NH₂).

(27) The process according to item (26), wherein said conversion step is carried out by catalytic hydrogenation in the presence of ammonia.

(28) The process according to any one of the preceding items, wherein methyl chloride is used in about 1 to 10 times molar amounts, preferably 2 to 5 times, more preferably 2.1 to 3 times relative to the compound of formula (II), or

wherein methyl chloride in gaseous form is used in 4 to 8 times molar amounts relative to the compound of formula (II) in volumes to 1 liter reaction mixture, preferably 2.5 to 4 times molar amounts relative to the compound of formula (II) in 1 to 10 liter reaction mixture, more preferably 2.20 to 3.60 molar amounts relative to the compound of formula (II) in more than 10 to less than 50 liters reaction mixture, and in particular 2.02 to 2.5 times molar amounts relative to the compound of formula (II) in reaction mixtures of 50 liters or more.

(29) The process according to any one of items (1) to (28), wherein methyl chloride is used in an excess of about between 2.0 to 2.2 times molar amounts relative to the compound of formula (II) in case the reaction is carried out in a closed vessel.

(30) The process according to any one of items item (1) to (29), wherein methyl chloride is used in liquid form in about 5 to 30 mass ratio excess and the reaction is performed at least at pressure which corresponds to the vapour pressure of methyl chloride at the temperature of the reaction.

(31) The process according to any one of the preceding items, wherein the proton acceptor is used in an amount of 2 to 4 molar amount, preferably 2.0 to 2.5 molar amount and more preferably 2.1 to 2.3 molar amount relative to the compound of formula (II).

(32) A process for preparing a compound comprising a dimethylated methylene group and further defined by having at least one group selected from the group consisting of cyclohexyl, —NH₂, —H₂NH₂, —CH₂NHR, —CH₂NR₂, —CHR′—NHR″, —CH₂OH, —CHR′—OH, COOH, wherein R is substituted or unsubstituted alkyl, substituted or unsubstituted aryl, substituted or unsubstituted arylalkyl, substituted or unsubstituted heteroaryl or substituted or unsubstituted heteroarylalkyl; and wherein R′ and R″ are selected from the group consisting of substituted or unsubstituted alkyl, substituted or unsubstituted aryl, substituted or unsubstituted arylalkyl, substituted or unsubstituted heteroaryl or substituted or unsubstituted heteroarylalkyl, comprising the steps of:
i) providing a compound of formula (I) prepared by a process according to claim 1 or 2, which comprises at least one of the W and Y groups being convertible by catalytic hydrogenation, and
ii) subjecting said W and/or Y group to catalytic hydrogenation.

According to this beneficial aspect of the invention, compounds being valuable intermediates for synthesis due to reactive groups like amino group, hydroxyl group or carboxylic acid group or due to the bulky cyclohexane group can be obtained in only few synthetic steps, while the reaction product of this process is advantageously pure, since there are little or no monomethyl and desmethyl impurities in step i). Due to the substantially full conversion in step i), no purification step of the product of step i) is necessary, and thus subsequent step ii) can be carried out with the crude product.

(33) The process according to item (32), wherein the compound obtained by said process comprises a COOH group and an electron withdrawing group W, wherein said COOH group is further subjected to decarboxylation subsequent to conversion of COOR to COOH.

According to this beneficial embodiment, monofunctional compounds are obtained, since the —COOH group will be replaced by —H after decarboxylation. These monofunctional compounds will be valuable precursors for the synthesis of therapeutic, prophylactic or diagnostic agents in which synthesis monofunctional precursors are necessary. On the other hand, if the present processes are applied for the preparation of intermediates for the synthesis of aliskiren or cryptophycin derivatives, conditions promoting decarboxylation of a product comprising a carboxylic acid group have to be avoided, because in the synthesis of aliskiren or cryptophycin derivatives, bifunctional intermediates are necessary/preferred.

(34) The process according to item (32) or (33), wherein the compound of formula (I) comprises at least one W and/or Y group(s) selected from the group consisting of phenyl, NO₂, N₃, cyano, CONHR, CONR₂, CR′═NR″, COOR, COR′, COOCH₂Ph wherein Ph is substituted or unsubstituted, wherein R is substituted or unsubstituted alkyl, substituted or unsubstituted aryl, substituted or unsubstituted arylalkyl, substituted or unsubstituted heteroaryl or substituted or unsubstituted heteroarylalkyl, and wherein R′ and R″ are independently selected from the group consisting of substituted or unsubstituted alkyl, substituted or unsubstituted aryl, substituted or unsubstituted arylalkyl, substituted or unsubstituted heteroaryl or substituted or unsubstituted heteroarylalkyl.

(35) The process according to any one of items (32) to (34), wherein methyl chloride as the methylating reagent of step i) and hydrogen as the hydrogenation reagent of step ii) are provided in gaseous form.

(36) A process for preparing 3-amino-2,2-dimethylpropanamide, comprising the steps of:
a) providing an ester or amide derivative of 2-cyano-2-methylpropanoic acid by the process according to item (8)
b) optionally converting Y being an ester group to amide group, and
c) converting W being a cyano-group to aminomethyl group (—CH₂—NH₂) by catalytic hydrogenation in the presence of ammonia.

(37) The process according to item (36), wherein step b) is carried out without performing a purification step of the product of step a).

(38) The process according to item (36) or (37), wherein the methyl chloride and hydrogen are introduced into the reaction in a gaseous state, optionally at elevated pressure.

(39) The process according to any one of items (36) to (38), further comprising subsequent reaction steps selected from a group of oxidation, reduction, alkylation, esterification, amidation, hydrolysis, cyclisation, deprotection and catalysis; or purification.

(40) The process according to item (39), wherein a therapeutic, prophylactic or diagnostic agent is obtained.

The term “therapeutic, prophylactic or diagnostic agent” as used herein means any active pharmaceutical ingredient intended for diagnosis, prophylaxis or treatment of any human or other mammal disease. In general it can mean any active pharmaceutical ingredient that has effect on the conditions of for example internal organs, blood circulation, growth, hormone levels, cell excretion, metabolism, or physiology, or can be used in tracking changes in said conditions. For example, therapeutic, prophylactic or diagnostic agent can mean antibiotic agent, antihypertension agent (like sartans, aliskiren, diuretics), hormones, vitamins, antidiabetic agents (like sulphonylureas, biguanides, thiazolidinediones), compound comprising radioactive iodine, or the like. The process according to the invention can be beneficially applied in the synthesis of such therapeutic, prophylactic or diagnostic agents.

(41) The process according to item (40), wherein said therapeutic, prophylactic or diagnostic agent is aliskiren or cryptophycin derivative, preferably aliskiren.

(42) The process according to item (40) or (41), comprising subsequent step(s) for obtaining a pharmaceutical dosage form comprising said therapeutic, prophylactic or diagnostic agent.

(43) A process for preparing therapeutic, prophylactic or diagnostic agent, wherein the process comprises the steps of:
a) providing a compound prepared by a process according to item (32), and
b) reacting said compound under conditions sufficient to produce a therapeutic, prophylactic or diagnostic agent.

(44) The process according to item (43), wherein said therapeutic agent is aliskiren or a cryptophycin derivative.

(45) A process for preparing aliskiren, wherein the process comprises the steps of:
a) providing a compound of formula (I)

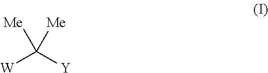

wherein W is CN and Y is COOR, CONH₂, CONHR or CONR₂, wherein R is substituted or unsubstituted alkyl, preferably methyl or ethyl, by the process according to item (8), and
b) reacting said compound of formula (I) under conditions sufficient to produce aliskiren or a pharmaceutically acceptable derivative thereof.

(46) A process for preparing cryptophycin derivatives, wherein the process comprises the steps of:
a) providing a compound of formula (I)

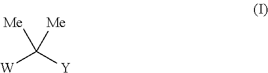

wherein W is CN and Y is COOP, wherein R is substituted or unsubstituted alkyl, preferably methyl or ethyl, by the process according to item (8), and
b) reacting said compound of formula (I) under conditions sufficient to produce a cryptophycin derivative or a pharmaceutically acceptable derivative thereof.

(47) A process for preparing aliskiren, wherein the process comprises the steps of:
a) providing 3-amino-2,2-dimethylpropanamide by the process according to item (36), and
b) reacting said 3-amino-2,2-dimethylpropanamide under conditions sufficient to produce aliskiren or a pharmaceutically acceptable derivative thereof.

(48) A process for preparing a cryptophycin derivative, wherein the process comprises the steps of:
a) providing 3-amino-2,2-dimethylpropanamide by the process according to item (36), and
b) reacting said 3-amino-2,2-dimethylpropanamide under conditions sufficient to produce a cryptophycin derivative or a pharmaceutically acceptable derivative thereof.

In the above defined processes for preparing aliskiren and cryptophycin respectively, it is particularly favorable to select the conditions as defined under items (1) to (39).

(49) Use of a compound prepared by the process according to any on of items (1) to (39) for the manufacture of a therapeutic, prophylactic or diagnostic agent.

(50) The use according to item (49), wherein the therapeutic, prophylactic or diagnostic agent is aliskiren or a cryptophycin derivative.

(51) Use of a compound of formula (I):

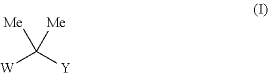

wherein W is CN and Y is COOR, CONH₂, CONHR or CONR₂, preferably COOR,
wherein R is substituted or unsubstituted alkyl, preferably methyl or ethyl,
prepared by the process according to item (8) for the manufacture of aliskiren.

(52) Use of a compound of formula (I):

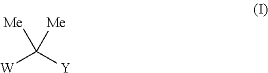

wherein W is CN and Y is COOR, wherein R is substituted or unsubstituted alkyl,
preferably methyl or ethyl,
prepared by the process according to item (9) for the manufacture of cryptophycin derivatives.

(53) Use of 3-amino-2,2-dimethylpropanamide prepared by the process according to any one of items (36) to (41) for the manufacture of aliskiren or cryptophycin derivatives, preferably aliskiren.

(54) Use of therapeutic, prophylactic or diagnostic agent obtained according to any one of items (43) to (48) for manufacture of a pharmaceutical dosage form.

(55) The process according to any one of items (1) to (31), wherein said process is carried out in a reaction mixture defined by one single liquid phase.

The term “one single liquid phase” as used herein means that there is no liquid-liquid interface in the liquid phase of the reaction mixture, that is there is only one liquid phase represented by the solvent and the components dissolved therein. In this way, fast or relatively fast reaction rates are provided since mass transport takes place in between one liquid phase only, that is there is no liquid-liquid interface impeding or even inhibiting mass transfer.

(56) The process according to any one of items (1) to (31) or (55), wherein said process is carried out without a phase transfer catalyst.

A phase transfer catalyst is for example a tertiary or quarternary alkylamine. According to this beneficial embodiment of the invention, in case the reaction mixture comprises an undissolved or partly undissolved solid component such as the proton acceptor and/or compound of formula (II), no phase transfer catalyst is required for providing or improving mass transport between the solid phase and the liquid phase.

In this invention it was surprisingly found that methyl chloride, even though it is under normal conditions in a gaseous state, is a very suitable methylation agent in dimethylation reaction of activated methylene groups. This is especially true when used in combination with an aprotic polar solvent. Surprisingly, methyl chloride has a very high solubility in said aprotic polar solvent, such that the losses in industrial scale are only minute even if the reaction takes place in a not tightly closed reactor. Another advantage surprisingly found was that methyl chloride is more reactive than methyl iodide in conditions disclosed herein, whereas methyl iodide is the more reactive methylating agent under conventional conditions. Thus, the dimethylation reaction of this invention runs until substantially no more starting material (desmethyl compound) or monomethylated compound is present.

The fact that methyl chloride is in a gaseous state under normal conditions, that is room temperature and atmospheric pressure, seemed at first an obstacle, as one needs proper pipe installation or adjusted reaction equipment to be able to introduce methyl chloride into the reaction mixture. Normally only well equipped laboratories or specifically industry have the appropriate equipment at their disposal. But with the present knowledge of the advantageous effects of methyl chloride, it is particularly welcome to introduce the aspects of the invention in a process for dimethylation of active methylene groups, since the low molecular weight methyl chloride is reasonably easy to handle in terms of storage and the possibilities of introducing it into the reaction mixture. Furthermore, methyl chloride is less toxic than methyl iodide or -bromide, and it is significantly cheaper compared to other methyl halogens. Thus, in view of the aforementioned advantages of methyl chloride, in industrial scale, the adaptation of the equipment for handling gaseous reactants will be welcomed.

An additionally observed advantage of using methyl chloride as the methylation agent in dimethylation reaction is the possibility of removing excess amounts of methyl chloride by bubbling the reaction mixture with other gas, preferably inert gas. This special feature provides for carrying out subsequent reaction steps in the same reaction mixture by simply adding further reagents, which further provides for significant savings of organic solvents. The present invention provides for improvements since the crude product can be used in subsequent steps without purification. In contrast to that, liquid methyl bromide and methyl iodide require the complete evaporation of solvent from the reaction mixture in order to eliminate unreacted methylation agent.

It was observed that using methyl chloride contributes to a more simplified process in cases when dimethylation reaction is preceding a catalytic hydrogenation reaction step. In such settings, two gaseous reactants instead of one are used. Methyl chloride can be introduced into the reaction mixture using the same pipe system used also for providing hydrogen into the reaction mixture. Methyl chloride is blown into the reaction in the same manner as hydrogen, demanding no extra modifications for using another gaseous reactant like methyl chloride. This makes use of already established equipment, changing the process to easy-to-handle, cheap, well controlled and with high yields. There can be intermediate reaction step(s) such as oxidation, hydrolysis, amidation, preferably amidation, applied after dimethylation and before advancing to catalytic hydrogenation. Optionally, the intermediate reaction step and catalytic hydrogenation step are combined to run simultaneously or subsequently, but as a one pot reaction. In conclusion, the present invention provides for a process comprising the combination of dimethylation and catalytic hydrogenation, wherein at least two gaseous reactants are used, preferably methyl chloride and hydrogen.

Choosing a solvent essentially consisting of a polar aprotic solvent or a mixture of a polar aprotic solvent and non-polar aprotic solvent (commonly referred to “aprotic solvent”) for the reaction further contributes to the advantageous effects according to the present invention. Aprotic solvent enables high solubility of methyl chloride. In addition, using aprotic solvent in dimethylating reaction together with methyl chloride provides for higher yields of dimethylated products being substantially free of nonmethylated or monomethylated products or other side products compared to dimethylation reactions wherein protic solvents are used. Aprotic solvent further contributes to the stability of methyl chloride in the reaction mixture, since methyl chloride is stable in aprotic solvent, while it would get quenched in the protic solvent. This feature again contributes to obtaining high yields of pure product.

### DETAILED DESCRIPTION OF THE INVENTION

The present invention relates to a dimethylation process of a compound of formula:

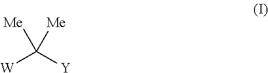

in which W denotes an electron withdrawing group having −M-effect,
Y is the same or different electron withdrawing group as W, or Y is selected from groups having +M-effect or no M-effect, except H,
wherein a compound of formula (II):

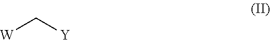

in which W and Y are defined as above, is reacted with methyl chloride in the presence of a proton acceptor.

The dimethylation process according the present aspect is suitable for substances comprising active methylene groups. The active methylene groups are methylene groups adjacent to one electron withdrawing group, preferably located between two electron withdrawing groups, which can be the same or different, making the hydrogen in the methylene groups more reactive. The electron withdrawing group W can be selected from a group consisting of CN, CHO, NO₂; COOR, CONH₂, CONHR, CONR₂ COSR, CSOR, CSNH₂, CSNHR and CSNH₂, where R is substituted or unsubstituted alkyl, substituted or unsubstituted aryl or substituted or unsubstituted arylalkyl, substituted or unsubstituted heteroaryl or substituted or unsubstituted heteroalkyl; COR′, SO₂R′, CR′═NR″, wherein R′ and R″ are independently selected from the group consisting of substituted or unsubstituted alkyl, substituted or unsubstituted aryl or substituted or unsubstituted arylalkyl, substituted or unsubstituted heteroaryl or substituted or unsubstituted heteroalkyl. Alternatively, electron withdrawing groups on both sides of the methylene group intended to be methylated can be bonded together to form a C4 to C8 ring, wherein W and Y cooperatively represent a group of the formula Z′(CH₂)ₚZ″, wherein Z′ and Z″ are the same or different and are CO, CO—O—, CO—NR*-, where R* is H, substituted or unsubstituted alkyl, substituted or unsubstituted aryl or substituted or unsubstituted arylalkyl, substituted or unsubstituted heteroaryl or substituted or unsubstituted heteroalkyl; CO—S—, and SO₂ group, and p is an integer between 1 and 4.

Said ring structure can comprise additional electron withdrawing groups or carbon atoms being replaced by oxygen, sulphur or nitrogen atoms.

Preferably, the electron withdrawing group W is selected from the group consisting of CN, NO₂; COOR, CONH₂, CONHR, CONR₂, COSR, CSOR, CSNH₂, CSNHR, CSNR₂ and COR, where R is substituted or unsubstituted alkyl, substituted or unsubstituted aryl, substituted or unsubstituted arylalkyl, substituted or unsubstituted heteroaryl or substituted or unsubstituted heteroalkyl.

The group Y in a substance according the formula (II) to be dimethylated is the same or different electron withdrawing group as W, or Y is selected from the group consisting of azido, substituted or unsubstituted aryl, substituted or unsubstituted alkyl, NHCOOR, SOR′, OR and SR′, preferably azido, substituted or unsubstituted aryl and substituted or unsubstituted alkyl, wherein R and R′ are selected from the group consisting of substituted or unsubstituted alkyl, substituted or unsubstituted aryl, substituted or unsubstituted arylalkyl, substituted or unsubstituted heteroaryl or substituted or unsubstituted heteroarylalkyl.

More preferably, the electron withdrawing group W is selected from the group consisting of COOR, CONH₂, CONHR, CONR₂, COSR, CSOR, CSNH₂, CSNHR, CSNR₂ and COR, where R is substituted or unsubstituted alkyl, substituted or unsubstituted aryl, substituted or unsubstituted arylalkyl, substituted or unsubstituted heteroaryl or substituted or unsubstituted heteroalkyl.

Particularly, the substances according the formula (II), wherein the electron withdrawing group W is selected from the group consisting of COOR, CONH₂, CONHR, CONR₂, COSR, CSOR, CSNH₂, CSNHR and CSNR₂, where R is substituted or unsubstituted alkyl, substituted or unsubstituted aryl, substituted or unsubstituted arylalkyl, substituted or unsubstituted heteroaryl or substituted or unsubstituted heteroalkyl, and Y is CN are selected. More particularly, the electron withdrawing group W is CN and Y is selected from the group consisting of COOR, CONH₂, CONHR, CONR₂, COSR, CSOR, CSNH₂, CSNHR, CSNR₂ and COR, wherein R is substituted or unsubstituted alkyl, substituted or unsubstituted aryl, substituted or unsubstituted arylalkyl, substituted or unsubstituted heteroaryl or substituted or unsubstituted heteroarylalkyl.

In a preferred embodiment, the compound according the formula (II), in which W is CN and Y is COOR, CONH₂, CONHR or CONR2, preferably COOR, wherein R is substituted or unsubstituted alkyl, preferably methyl or ethyl, is dimethylated with methyl chloride in the presence of a proton acceptor in a solvent essentially consisting of a polar aprotic solvent or a mixture of a polar aprotic solvent and non-polar aprotic solvent. According to a further preferred embodiment, the electron withdrawing group W is CN and Y is COOR wherein R is substituted or unsubstituted alkyl or benzyl, preferably methyl, ethyl or benzyl, more preferably methyl or ethyl.

According to another embodiment of the invention, dimethylation of active methylene groups is performed in a solvent essentially consisting of a polar aprotic solvent or a mixture of a polar aprotic solvent and non-polar aprotic solvent.

Said solvent is preferably used in a mass ratio of solvent to compound of formula (II) of about 1 to about 20, preferably 1 to 5. In industrial scale, the amount of solvent should be as low as possible, but it is limited by the viscosity of the reaction mixture. The above mentioned range for the amount of solvent provides for setting the viscosity of the reaction mixture within an advantageous range which enables a sufficient agitation and thus a reliable and robust process. The more preferred mass ratio of solvent to compound of formula (II) is between about 2 to about 3.

However, it may be possible to reduce the amount of solvent to a mass ratio of solvent to compound of formula (II) of less than 1. In this way, it is advantageous to save organic solvents, which contributes to environmental friendliness, working conditions and possibly economy of said process. The amount of solvent to be used in the above process depends on the solubility of compound of formula (II) within said solvent. In case compound of formula (II) is very readily soluble in said solvent or compound of formula (II) is a liquid, the preferred embodiment may be applied, wherein even a lower amount of solvent can be used in a mass ratio of solvent to compound of formula (II) of less than 0.5, preferably less than 0.3, more preferably less than 0.1. According to another embodiment, said process can be even carried out in the absence of solvent. This embodiment is applicable to compounds of formula (II) which are in liquid or fluid state (as illustrated, for example by Example 2). Preferably, in this embodiment, the liquid compound of formula (II) and an excess of liquid methyl chloride is used in order to guarantee sufficiently low viscosity to carry out the reaction without solvent. The excess of liquid methyl chloride is preferably 5 to 30 mass ratio, most preferably 8 to 15. Liquid methyl chloride should be mixed with other compounds at temperature lower than its boiling point, the mixture is then tightly closed to reaction container and warmed to the reaction temperature. The pressure follows the vapour pressure of methyl chloride at the corresponding temperature. The excess of chlorinating agent which evaporates after opening the reaction vessel is simply collected in a freezing condenser and used in a following batch. Such process is even more feasible in industrial scale than in a laboratory. The opportunity of complete omission of the solvent, makes the process especially advantageous in view of environmental friendliness, working conditions and possibly economy of said process.

The proton acceptor to be used in a further preferred embodiment can be any substance of pKa over about 8, preferably of pKa from about 8 to about 12. For example, proton acceptors like basic substances, especially inorganic bases such as sodium hydride, alkali or earth alkali hydroxides, preferably sodium hydroxide, or alkoxides, preferably sodium alkoxide can be used. However, the preferred embodiments involve dimethylation of compounds comprising ester, amide or thioester groups, rendering strong proton acceptors unsuitable for the process, as the starting compound is subjected to hydrolysis or transesterification. Instead, mild proton acceptors are to be chosen in this case. Best results are achieved when using alkali metal carbonates. Preferably selected are caesium carbonate, lithium carbonate, rubidium carbonate, sodium carbonate and potassium carbonate, more preferably caesium carbonate and potassium carbonate, yet more preferably potassium carbonate.

The advantage of using potassium carbonate over caesium carbonate in the present process resides in the fact that the caesium carbonate represents the carbonate with the larger cation. Carbonates with bigger counter-cation are far more dissociated in aprotic solvents and are more soluble in the aprotic solvents, therefore it is more difficult to remove them later after the reaction is completed. The solubility of caesium carbonate at ambient temperature in N,N-dimethylformamide (DMF) and dimethylsulfoxide (DMSO) is 1.195 g/10 mL and 3.625 g/10 mL, respectively, whereas the solubility of potassium carbonate in the same solvents is 0.075 g/10 mL and 0.470 g/10 mL, respectively. The solubility of potassium carbonate is sufficient for enabling the dimethylation reaction, but at the same time in the case of any reaction step, simple filtration is enough to remove most of potassium carbonate in order to enable a smooth transfer of the reaction to the next synthetic step, e.g. hydrogenation. In contrast to strong proton acceptors, potassium carbonate has the advantage that it does not hydrolyse starting compounds comprising ester, amide or carbamate groups, and does not hydrolyse obtained products when water addition is needed to isolate them.

According to another embodiment, the process involves providing a compound with active methylene group in the polar aprotic solvent or a mixture of a polar aprotic solvent and additional aprotic solvent before, together or after providing the proton acceptor in the solvent, wherein the proton acceptor is preferably alkali metal carbonate, more preferably caesium carbonate and potassium carbonate, yet more preferably potassium carbonate. In addition, methyl chloride is added to the solvent or the reaction mixture independently of the other components, preferably after the compound with active methylene group and a proton acceptor have been added to the solvent. Methyl chloride is added to the reaction in any aggregate state, meaning it can be cooled to the liquid and added, but preferably it is added in a gaseous state. The addition of methyl chloride can be in one portion, in multiple portions or continuous. The most preferred option is a continuous addition during a period of time of multiple hours, preferably about five hours.

In a preferred embodiment of the present invention, the polar aprotic solvent is selected from a group of sulfoxides, most preferably DMSO, sulphones most preferably sulfolane, and amides, preferably from N,N-dimethylformamide, N,N-dimethylacetamide, N-methylpyrrolidone, hexamethylphosphortriamide, 1,1,3,3-tetramethylurea or 1,3-dimethyl-3,4,5,6-tetrahydro-2-(1H)-pyrimidinone, more preferably from N,N-dimethylformamide, or N,N-dimethylacetamide, most preferably from N,N-dimethylformamide.

According to present embodiment, polar aprotic solvent can be used alone or in a mixture of various polar aprotic solvents. Optionally, the polar aprotic solvent is used in a mixture with non-polar aprotic solvent, possibly selected from acetonitrile, ethers or hydrocarbons, preferably acetonitrile, diethylether, THF, pentane and hexane. The amount of non-polar aprotic solvent is limited, since such solvents decrease the solubility of the proton acceptor, which in turn results in decreased conversion and thus in decreased reaction yields. Thus, a solvent essentially consisting of a mixture of a polar aprotic solvent and a non-polar aprotic solvent having a volume ratio of polar aprotic solvent to non-polar aprotic solvent of 1:0 to 1:2 is used, and preferably said ratio is selected with the proviso that sufficient solubility of a proton acceptor is provided. More preferably, the non-polar solvent is added only in order to enhance the solubility of the starting compound of formula (II) or to optimize the reaction yield.

The process is carried out at atmospheric pressure or at elevated pressure, preferably at pressures from about 1 to about 3 bars, more preferably at atmospheric pressure.

Methyl chloride can be used in equimolar amounts with respect to the compound comprising active methylene group, just in double molar amount, or in molar excess of 2.1 times molar amount, 2.5 times, 3 times, 5 times and 10 times molar amount, or in excess of even over 10 times molar amount. Because methyl chloride is a gas, the required excess in opened vessels depends on losses of evaporation and is highly dependent from the volume of the reaction mixture. The preferred molar excess of methyl chloride is 4 to 8 times molar amounts relative to the compound of formula (II) in volumes to 1 liter, more preferably 2.20 to 3.60 in more than 10 to less than 50 liters reaction mixture, and in particular 2.5 to 4 times molar amounts relative to the compound of formula (II) in 1 to 10 liter volume and 2.02 to 2.5 molar amounts relative to the compound of formula (II) in industrial volumes which are at least 50 liters. Surprisingly, methyl chloride has a very high solubility in said aprotic solvents, therefore, the losses in industrial scale are negligible even if the reactor is not tightly closed. In tightly closed vessels, especially under higher pressure, the excess of between 2.0 and 2.2 times molar amounts relative to the compound of formula (II) is usually sufficient to complete the reaction and to bring the remainder of the monomethylated byproduct to far below 1 molar %.

The reaction mixture can be analyzed by gas chromatography (GC) and the reaction stopped when the concentration of the monomethylated intermediate drops down below 1 area % compared to the dimethylated product, preferably below 0.1%, most preferably below the limit of detection. Usually this takes from about 5 to about 48 hours, preferably about 12 to about 18 hours.

The reaction can be carried out at a temperature from about −10° C. to about 100° C., preferably from about 15 to about 35° C. in opened vessels or at atmospheric pressure. At elevated pressures, the temperature of the reaction might be considerably lower than room temperature, preferably below about 0° C.

Contrary to methyl sulphate and even contrary to many methods of dimethylation with methyl iodide, the dimethylation of cyanoacetates with methyl chloride according to conditions of the present invention runs until substantially no desmethylated or monomethylated substrate is present. In comparative example 1 wherein dimethyl sulphate is used as methylation agent, 25 to 35% of monomethyl analogue remains in the reaction mixture. In case of comparative example 1, the yield of the dimethylated product could be improved by elevating temperature, but the reaction mixture becomes instable and changes color considerably. Besides the problems of incompleteness of dimethylation by dimethyl sulphate, methyl chloride is superior in the possibility of completely removing its excess, either by bubbling with inert gas or heating the solution, while dimethyl sulphate lets oily residues which can seriously limit the use of crude products in further chemical conversions. With regard to reaction efficiency, similar observation could be made when using methyl iodide, which is disclosed in comparative example 2. After 20 hours of stirring the complete reaction mixture it still contained at least 8% of insufficiently reacted starting material. In contrast to that, when using methyl chloride according to the concept of the present invention, substantially no trace of the desmethylated or monomethylated residuals are left (as illustrated, for example by Examples 1 and 3) wherein the amount of monomethylated compound was analyzed by GC).

The obtained dimethylated product is isolated by any conventional chemical method, but the preferred method includes a filtration of inorganic precipitates, washing the precipitate by an organic solvent and water, preferably the same one as is used in the extraction. The product is isolated by treating the collected filtrates by two phase solvent/water system, removing of water phase and evaporating the organic solvent. The crude product can be further purified by conventional chemical methods such as distillation for liquid products, recrystallization for solid compounds or by chromatography as a general purification method. If reaction setup allows, the preferred option is to use the crude product for further subsequent chemical conversion, and preferably the subsequent reaction is carried out in the same solvent.

An ester derivative of 2-cyano-2-methylpropanoic acid, preferably methyl or ethyl ester, prepared by dimethylation with methyl chloride, preferably crude ester without special purification can be converted to amide by treating it with ammonia, preferably diluted with an alcohol, most preferably with methanol at temperatures from the boiling point of liquid ammonia to 100° C., preferably at room temperature for about 5 to about 48 hours, more preferably for about 12 to about 18 hours, to give 2-cyano-2-methylpropanamide, which is isolated by conventional chemical methods. Crude product is optionally purified by recrystallization from a solvent, most preferably from isopropanol.

Alternatively, 2-cyano-2-methylpropanamide can be prepared by dimethylation of cyanoacetamide with methyl chloride according to the process of the present invention. In this case, the amide derivative of 2-cyano-2-methylpropanoic acid, that is cyanoacetamide, can be directly subjected to conversion of the cyano-group to aminomethyl group.

The cyano group of a dimethylated compound comprising a cyano group obtained according to the present invention can be converted before or after conversion of the other electron withdrawing group, like for example an ester group which is converted to amide group. This can be done by catalytic hydrogenation reduction, where the presence of the catalyst and ammonia or amine is required. The suitable catalyst would be easily identified by the person skilled in the art. In general, the catalyst may be for example sponge catalyst, supported catalyst, thin-layer catalyst or unsupported catalyst. Preferably the catalyst comprise at least one noble metal like palladium, cobalt, platinum or nickel. In addition, it can optionally comprise at least one metal from the group of copper, manganese, chromium and iron. Preferably, the hydrogenation is performed on Raney cobalt catalyst or Raney nickel catalyst, more preferably on Raney nickel catalyst. With regard to the presence of ammonia and amine, they can be used either alone or in a combination. However, better results are achieved when using only one, in particular ammonia. Suitable amines for use in the present invention are in particular mono- or dialkylamines, especially methyl- or dimethylamine. The catalytic hydrogenation in the presence of the catalyst and ammonia or amine is conducted at elevated temperature from about 25 to about 100° C., preferably from about 70 to about 80° C., in a solvent selected preferably from alcohols, most preferably methanol. The final product is isolated by conventional chemical methods, preferably by recrystallization.

In a preferred embodiment, 2-cyano-2-methylpropanamide is converted to 3-amino-2,2-dimethylpropanamide. The invention provides an industrial process for preparing 3-amino-2,2-dimethylpropanamide comprising reacting methyl cyanoacetate with methyl chloride in the presence of alkali metal carbonate in a solvent essentially consisting of a polar aprotic solvent or a mixture of a polar aprotic solvent and non-polar aprotic solvent, converting ester group to amide group, and converting cyano-group to amine by catalytic hydrogenation using hydrogen in the presence of ammonia or amine, wherein the methyl chloride and hydrogen are introduced into the reaction in a gaseous state, optionally at elevated pressure. A further alternative is to advance the dimethylation of methyl cyanoacetate by converting ester group to amide group and converting cyano-group to amine simultaneously in one pot. This can be done by applying special reaction conditions, like for example high pressure and increased temperature. Pressure should be raised up to 2-10 bars and the temperature is preferably set between 20° C. to 150° C. It is understood that the 3-amino-2,2-dimethylpropanamide according to the present invention can be obtained without the need of intermediate of simultaneous conversion of ester group to amide group when commencing from cyano acetamide.

The dimethylation of compounds according to the present invention is, besides converting ester group to amide group and/or reducing the cyano group, easily tied to further conversions. The conversion can comprise additional chemical reactions such as for example oxidation, reduction, alkylation, esterification, amidation, hydrolysis, cyclisation, deprotection or catalysis; or purification, in order to obtain therapeutic, prophylactic or diagnostic agent, preferably aliskiren or cryptophycin derivatives. Particularly, dimethylated compounds according to the present invention can be used as intermediates in the route of synthesis of therapeutic, prophylactic or diagnostic agent. Specifically they can be used for preparing aliskiren or cryptophycins. In a special example methyl cyanoacetate is reacted with methyl chloride in the presence of a proton acceptor in a solvent essentially consisting of a polar aprotic solvent or a mixture of a polar aprotic solvent and non-polar aprotic solvent, which proceeds with the conversion of the ester group to amide group, which proceeds with the conversion of the cyano group to aminomethyl group (—CH₂—NH₂) to obtain 3-amino-2,2-dimethylpropanamide, wherein thus obtained 3-amino-2,2-dimethylpropanamide is converted to obtain aliskiren. Again, conversion of ester group to amide group can be done first and cyanoacetamide is used as a starting material for dimethylation. It would be understood that also ethyl cyanoacetate or other alkyl cyanoacetate can be optionally used. This has a bearing that crude ester derivatives of 2-cyano-2-methylpropanoic acid can be prepared according the present invention and used to prepare 3-amino-2,2-dimethylpropanamide, which can be further used as a building block in preparation of for example antihypertensive aliskiren. Further teaching for making of aliskiren by using 3-amino-2,2-dimethylpropanamide substantiated with necessary examples can be found in EP 0678503. Similarly, ester derivatives of 2-cyano-2-methylpropanoic acid can be used to prepare 3-amino-2,2-dimethylpropanoate, which can in turn be applied in preparing anticancer drug cryptophycins. The necessary teaching of the synthesis of anticancer cryptophycins is disclosed in WO 00/023429.

The therapeutic, prophylactic or diagnostic agent, preferably aliskiren or cryptophycin derivatives, more preferably aliskiren, obtained by converting the dimethylated compound prepared according to the present invention can be administered to humans or other mammals. For example, administration can be oral, parenteral (subcutaneous, intravenous, intramuscular, intraperitoneal) or topical. Alternatively, or concurrently, the administration can be by air passage route. The therapeutic, prophylactic or diagnostic agent can be used either alone or in combination with other therapeutic agents. They can be administered alone or together with pharmaceutically acceptable excipients, which would be selected on the basis of the chosen route of administration and acknowledged pharmaceutical practice. In both instances, the therapeutic, prophylactic or diagnostic agent prepared according to the present invention, alone or in combination, would be adapted for administration, which in general terms means it would be administered as a pharmaceutical dosage form. Dosage form can be selected according to the proper route of administration, but would in general be selected from a group of oral solid dosage forms, such as tablets, capsules, granules, pellets, powders; liquid dosage forms such as syrups, suspensions, emulsions, solutions; and semisolid dosage forms such as creams, ointments, foams or the like. Depending on the need, the dosage forms can be prepared as sterile or otherwise adapted for administration; for example, pellets or tablets can be filled in capsules, tablets can be coated, instable suspension can be converted into stable ones, or the like.

For preparation of the dosage forms comprising therapeutic, prophylactic or diagnostic agent, preferably pharmaceutical acceptable excipients are used. For example, diluents like lactose, starch, or cellulose derivatives, glidants like talk, magnesium stearate and stearic acid, desintegrators like croscarmelose sodium, binders like gelatine, polyethylene glycol or the like are used for solid dosage forms. Water, suitable oils, saline, dextrose, propylene glycol or polyethylene glycol, EDTA, salts, antioxidizing agents (sodium bisulfite, ascorbic acid) or the like can be used to prepare liquid dosage forms. For semisolid dosage forms, water and oils, together with stabilizing agent, antioxidizing agent, or the like can be used for preparation. Other pharmaceutically acceptable excipients will be immediately apparent to the skilled person.

Thus, the process of the present invention can comprise further step(s) of obtaining a pharmaceutical dosage form, comprising therapeutic, prophylactic or diagnostic agent, preferably aliskiren or cryptophycins, more preferably aliskiren. The dosage of the therapeutic, prophylactic or diagnostic agent administered, preferably aliskiren or cryptophycins, more preferably aliskiren, depends on the age, health and condition of the recipient, taking into consideration also any concurrent treatment and desired effect to be achieved, all of which would be apparent to the skilled person. It can vary from submilligram doses to more than 100 milligram-, 500 milligram- or even over 1000 milligram-doses. To prepare a medicament, prepared dosage forms are packed in suitable package like for example blisters, plastic or glass bottles, vials, syringes, sacks, or the like.

The following examples are merely illustrative of the present invention and they should not be considered as limiting the scope of the invention in any way, as these examples, modifications and other equivalents thereof will become apparent to those versed in the art in the light of the present disclosure and the accompanying claims. Reactions are followed by GC chromatography and the ratio among starting compounds, intermediates are defined as the ratio of peak areas.

### Example 1 (According to the Invention)

### Preparation of methyl 2-cyano-2-methylpropanoate (in DMF as the aprotic polar solvent)

Methyl chloride was slowly added into the stirring mixture of methyl cyanoacetate (198 g), potassium carbonate (607.2 g) in 500 ml of DMF at temperature 15-30° C. Kinetics was checked by gas chromatography (GC). After about 374 g of methyl chloride was added (approx. 5 h) there was still 20% of monomethyl derivative. Stirring and adding methyl chloride (with reduced flow) at 15-30° C. was continued until GC showed monomethyl derivative dropped below 0.1% area (usually there was no more detectable monomethyl derivative). The total consumption of methyl chloride was 400 g. Total reaction time varies from 12-18 h.

Reaction mixture was then bubbled by nitrogen, solid material was filtered and the filter cake was washed with 800 ml of methyl t-butyl ether (MTBE). Filtrates were then washed with 800 ml of water. Water phase was again extracted with 270 ml of MTBE. The combined organic phase was washed twice with 500 ml of 5% NaCl and evaporated to obtain 222.8 g (88%) of crude methyl 2-cyano-2-methyl propanoate in the form of brown-yellow oil which was used in next step without purification.

### Example 2 (According to the Invention)

### Preparation of methyl 2-cyano-2-methylpropanoate (in the absence of a solvent)

In a stainless steel high pressure vessel equipped by anchor like stirrer, finely pulverized potassium carbonate (1.21 kg) is mixed with methyl cyanoacetate (0.4 kg) by vigorous stirring for 2 hours to obtain thick milky suspension. Then, the mixture is cooled to −30° C., followed by cautious addition of liquid methyl chloride (3.5 L). The vessel is then tightly closed up, the reaction mixture is warmed to 35° C. and vigorously stirred for 36 h. Finally, the pressure is reduced by opening a valve and the access of methyl chloride is distilled to a low temperature condenser by keeping the temperature of the mixture between 10-20° C. and with gradual addition of water (2 L). After most of methyl chloride is removed, the upper layer is separated and water phase is washed twice with 0.5 L of methyl t-butyl ether. The combined organic phase are washed twice with 250 ml of 5% NaCl and evaporated to obtain 452 g of crude methyl 2-cyano-2-methyl propanoate which is distilled to form 412 g (80%) of slightly yellow coloured oil.

### Example 3 (According to the Invention)

### Preparation of ethyl 2-cyano-2-methylpropanoate (in DMF as the aprotic polar solvent)

Methyl chloride was slowly added into the stirring mixture of ethyl cyanoacetate (113 g), potassium carbonate (303.6 g) in 500 ml of DMF at temperature 15-30° C. Kinetics was followed by GC. After about 195 g of methyl chloride was added (approx. 5 h) there was still 23% of monomethyl derivative. Stirring and adding methyl chloride (with reduced flow) at 15-30° C. was continued until GC showed monomethyl derivative dropped below 0.1% area (usually there was no more detectable monomethyl derivative). The total consumption of methyl chloride was 220 g.

Reaction mixture was then bubbled by nitrogen, solid material was filtered and the filter cake was washed with 750 ml of MTBE. Filtrates were then washed with 400 ml of water. Water phase was again extracted with 250 ml of MTBE. The combined organic phase was washed twice with 250 ml of 5% NaCl and evaporated to obtain 108.0 g (85%) of crude ethyl 2-cyano-2-methylpropanoate in the form of brown-yellow oil which was used in next step without purification.

### Comparative Example 1

### Preparation of ethyl 2-cyano-2-methylpropanoate

Mixture of ethyl cyanoacetate (5.65 g), potassium carbonate (13.8 g) in 50 ml of DMF was cooled to 10° C., and then 15.75 g of methyl sulphate was slowly added within 0.5 h while temperature was maintained below 35° C. Stirring was continued for 18 h at room temperature and the resulting suspension was filtered, washed with 70 ml of MTBE. Combined filtrate were then washed with water (50 ml), water phase was again extracted with 30 ml of MTBE, organic phase was added to first crops of extracts and the combined fractions were finally washed twice with 30 ml of 5% NaCl and evaporated to obtain 5.2 g of product in a form of brown oil which showed 32.5% (GC, area) of monomethyl derivative and some amounts (6%) of unidentified impurities.

### Comparative Example 2

### Preparation of ethyl 2-cyano-2-methylpropanoate

Methyl iodide (49.6 ml, 50% access) was slowly added into the stirring mixture of ethyl cyanoacetate (30 g), potassium carbonate (73.4 g) in 80 ml of DMF keeping temperature below 30° C. The mixture was stirred for further 20 h at room temperature, salts were filtered, washed by fresh MTBE. The filtered solution was washed by 120 ml of 0.1 N HCl, 120 ml of brine and evaporated to give 35 g of solid title product which contained 8 area % of monomethylated impurity measured by GC.

### Example 4 (According to the Invention)

### Preparation of 2-cyano-2-methylpropanamide

Crude methyl 2-cyano-2-methyl propionate from example 1 was dissolved in 700 ml of methanol-ammonia mixture (169 g of NH₃ per kg) and stirred at room temperature for 15 h. Solvent was then evaporated and the remaining crude product was crystallized from 600 ml of isopropanol to give 159.6 g (81%) of white crystals.

### Example 5 (According to the Invention)

### Preparation of 3-amino-2,2-dimethylpropanamide

Product of Example 4 was transferred into autoclave after dissolved in 840 ml of methanol-ammonia mixture (169 g of NH₃ per kg), and then 47.9 g of Raney Ni was added. The mixture was hydrogenated while stirred for 10 h at 60-70° C. and at 5 bar of hydrogen. When analysis showed no more starting material, reaction mixture was filtered, washed with methanol and evaporated to give 160 g (97%) of crude titled product. which was further recrystallized from the 800 ml of isopropanol-toluene (1:9). Total yield of the experiment was 124 g (78%).

### Example 6 (According to the Invention)

### Preparation of Aliskiren

Product of example 6 is further converted according to the teaching of EP 0678503 to obtain aliskiren.

1. A process for preparing a compound of the formula (I): 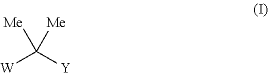
in which W denotes an electron withdrawing group having −M-effect,
Y is the same or different electron withdrawing group as W, or Y is selected from groups having +M-effect or no M-effect, except H,
wherein a compound of formula (II): 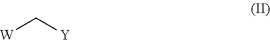
in which W and Y are defined as above, is reacted with methyl chloride in the presence of a proton acceptor in a solvent essentially consisting of a polar aprotic solvent or a mixture of a polar aprotic solvent and non-polar aprotic solvent.

2. A process for preparing a compound of the formula (I): 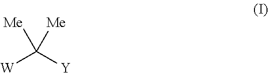
in which W denotes an electron withdrawing group having −M-effect,
Y is the same or different electron withdrawing group as W, or Y is selected from groups having +M-effect or no M-effect, except H,
wherein a compound of formula (II): 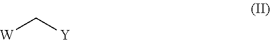
in which W and Y are defined as above, is reacted with methyl chloride in the presence of a proton acceptor in the absence of a solvent.

3. The process according to claim 1 or 2, wherein W is selected from the group consisting of:
CN, CHO and NO₂;
COOR, CONH₂, CONHR, CONR₂ COSR, CSOR, CSNH₂, CSNHR and CSNR₂, wherein R is substituted or unsubstituted alkyl, substituted or unsubstituted aryl, substituted or unsubstituted arylalkyl, substituted or unsubstituted heteroaryl or substituted or unsubstituted heteroarylalkyl; and
COR′, SO₂R′, CR′═NR″, wherein R′ and R″ are selected from the group consisting of substituted or unsubstituted alkyl, substituted or unsubstituted aryl, substituted or unsubstituted arylalkyl, substituted or unsubstituted heteroaryl and substituted or unsubstituted heteroarylalkyl;
or W and Y cooperatively represent a group of the formula Z′(CH₂)ₚZ″, wherein Z′ and Z″ are the same or different and are either CO, CO—O—, CO—NR*-, CO—S—, and SO₂ group, wherein R* is H, substituted or unsubstituted alkyl, substituted or unsubstituted aryl, substituted or unsubstituted arylalkyl, substituted or unsubstituted heteroaryl or substituted or unsubstituted heteroarylalkyl, and p is an integer between 1 and 4; and Y is the same or different electron withdrawing group selected from W defined above, or Y is selected from the group consisting of azido, substituted or unsubstituted aryl, substituted or unsubstituted alkyl, NHCOOR, SOR′, OR′ and SR′, substituted or unsubstituted aryl and substituted or unsubstituted alkyl, wherein R and R′ are selected from the group consisting of substituted or unsubstituted alkyl, substituted or unsubstituted aryl, substituted or unsubstituted arylalkyl, substituted or unsubstituted heteroaryl and substituted or unsubstituted heteroarylalkyl; or
wherein W is selected from the group consisting of:
CN and NO₂;
COOR, CONH₂, CONHR, CONR₂, COSR, CSOR, CSNH₂, CSNHR, CSNR₂ and COR,
wherein R is substituted or unsubstituted alkyl, substituted or unsubstituted aryl, substituted or unsubstituted arylalkyl, substituted or unsubstituted heteroaryl or substituted or unsubstituted heteroarylalkyl; and Y is the same or different electron withdrawing group selected from W defined above, or Y is selected from the group consisting of azido, substituted or unsubstituted aryl, substituted or unsubstituted alkyl, NHCOOR, SOR′, OR,′ SR′, substituted or unsubstituted aryl and substituted or unsubstituted alkyl, wherein R and R′ are selected from the group consisting of substituted or unsubstituted alkyl, substituted or unsubstituted aryl, substituted or unsubstituted arylalkyl, substituted or unsubstituted heteroaryl and substituted or unsubstituted heteroarylalkyl; or
wherein W is selected from a group consisting of:
COOR, CONH₂, CONHR, CONR₂, COSR, CSOR, CSNHR, CSNH₂, CSNR₂ and COR,
wherein R is substituted or unsubstituted alkyl, substituted or unsubstituted aryl, substituted or unsubstituted arylalkyl, substituted or unsubstituted heteroaryl or substituted or unsubstituted heteroarylalkyl; and Y is the same or different electron withdrawing group selected from W defined above, or Y is selected from the group consisting of azido, substituted or unsubstituted aryl, substituted or unsubstituted alkyl, NHCOOR, SOR′, OR,′ and SR′, substituted or unsubstituted aryl and substituted or unsubstituted alkyl, wherein R and R′ are selected from the group consisting of substituted or unsubstituted alkyl, substituted or unsubstituted aryl, substituted or unsubstituted arylalkyl, substituted or unsubstituted heteroaryl Of and substituted or unsubstituted heteroarylalkyl; or
wherein W is selected from a group consisting of: COOR, CONH₂, CONHR, CONR₂, COSR, CSNH₂, CSNHR and CSNR₂, wherein R is substituted or unsubstituted alkyl, substituted or unsubstituted aryl, substituted or unsubstituted arylalkyl, substituted or unsubstituted heteroaryl or substituted or unsubstituted heteroarylalkyl, and Y is CN; or
wherein W is CN and Y is selected from a group consisting of COOR, CONH₂, CONHR, CONR₂, COSR, CSOR, CSNH₂, CSNHR, CSNR₂ and COR, wherein R is substituted or unsubstituted alkyl, substituted or unsubstituted aryl, substituted or unsubstituted arylalkyl, substituted or unsubstituted heteroaryl or substituted or unsubstituted heteroarylalkyl; or
wherein W is CN and Y is COOR wherein R is substituted or unsubstituted alkyl or benzyl.

4. The process according to claim 3, wherein W is CN and Y is COOR, CONHR, CONH₂, or CONR₂, wherein R is substituted or unsubstituted alkyl.

5. The process according to claim 3, wherein the proton acceptor is selected from the group of alkali metal carbonates consisting of lithium, sodium, cesium and potassium carbonate.

6. The process according to claim 1, wherein compound of formula (II) is reacted in a solvent essentially consisting of a polar aprotic solvent or a mixture of a polar aprotic solvent and non-polar aprotic solvent;
wherein the polar aprotic solvent is selected from the group consisting of sulfoxides, sulphones and amides.

7. The process according to claim 3, wherein said process is carried out in a reaction mixture defined by one single liquid phase.

8. The process according to claim 3, wherein said process is carried out without a phase transfer catalyst.

9. A process for preparing a compound comprising a dimethylated methylene group and further defined by having at least one group selected from the group consisting of cyclohexyl, —NH₂, —CH₂NH₂, —CH₂NHR, —CH₂NR₂, —CHR′—NHR″, —CH₂OH, —CHR′—OH, and COOH, wherein R is substituted or unsubstituted alkyl, substituted or unsubstituted aryl, substituted or unsubstituted arylalkyl, substituted or unsubstituted heteroaryl or substituted or unsubstituted heteroarylalkyl; and wherein R′ and R″ are selected from the group consisting of substituted or unsubstituted alkyl, substituted or unsubstituted aryl, substituted or unsubstituted arylalkyl, substituted or unsubstituted heteroaryl and substituted or unsubstituted heteroarylalkyl, comprising the steps of:
i) carrying out the process according to claim 1 or 2, wherein at least one of the W and Y groups of the prepared compound of formula (I) is convertible by catalytic hydrogenation, and
ii) subjecting said W and/or Y group to catalytic hydrogenation.

10. A process for preparing 3-amino-2,2-dimethylpropanamide, comprising the steps of:
a) carrying out the process according to claim 4, wherein the prepared compound of formula (I) is an ester or amide derivative of 2-cyano-2-methylpropanoic acid,
b) optionally converting Y being an ester group to amide group, and
c) converting W being a cyano-group to aminomethyl group (—CH₂—NH₂) by catalytic hydrogenation in the presence of ammonia.

11. A process for preparing therapeutic, prophylactic or diagnostic agent, wherein the process comprises the steps of:
a) carrying out the process according to claim 9 or 10, and
b) reacting the compound prepared in step a) under conditions sufficient to produce a therapeutic, prophylactic or diagnostic agent.

12. A process for preparing aliskiren, wherein the process comprises the steps of:
a) carrying out the process according to claim 4, wherein the prepared compound is a compound of formula (I) 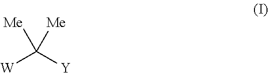
wherein W is CN and Y is COOR, CONH₂, CONHR or CONR₂, wherein R is substituted or unsubstituted alkyl, preferably methyl or ethyl, and
b) reacting said compound of formula (I) under conditions sufficient to produce aliskiren or a pharmaceutically acceptable derivative thereof.

13. A process for preparing a cryptophycin derivative, wherein the process comprises the steps of:
a) carrying out the process according to claim 4, wherein the prepared compound is a compound of formula (I) 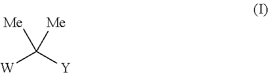
wherein W is CN and Y is COOR, wherein R is substituted or unsubstituted alkyl, preferably methyl or ethyl, and
b) reacting said compound of formula (I) under conditions sufficient to produce a cryptophycin derivative or a pharmaceutically acceptable derivative thereof.

14. A process for preparing aliskiren, wherein the process comprises the steps of:
a) carrying out the process according to claim 10, and
b) reacting the prepared 3-amino-2,2-dimethylpropanamide under conditions sufficient to produce aliskiren or a pharmaceutically acceptable derivative thereof.

15. A process for preparing a cryptophycin derivative, wherein the process comprises the steps of:
a) carrying out the process according to claim 10, and
b) reacting the prepared 3-amino-2,2-dimethylpropanamide under conditions sufficient to produce a cryptophycin derivative or a pharmaceutically acceptable derivative thereof.

16-19. (canceled)
